(19) United States
(12) Patent Application Publication
(10) Pub. No.: US 2024/0091435 A1
(43) Pub. Date: Mar. 21, 2024

(54) MECHANISM PROVIDING VARIABLE FILL CAPABILITY FOR A LIQUID RESERVOIR AND PUMP

(71) Applicant: INSULET CORPORATION, Acton, MA (US)

(72) Inventors: Maureen MCCAFFREY, Arlington, MA (US); Soroush KAMRAVA, Everett, MA (US); Steven CARDINALI, Tewksbury, MA (US); Jeffrey BARNES, Medford, MA (US); Kyle BREINGAN, Lowell, MA (US); Lucas MCGAHREN, Somerville, MA (US); Daniel BARRETT, Somerville, MA (US)

(21) Appl. No.: 18/466,217

(22) Filed: Sep. 13, 2023

Related U.S. Application Data
(60) Provisional application No. 63/375,986, filed on Sep. 16, 2022.

(51) Int. Cl. A61M 5/145; F04B 17/00

(52) U.S. Cl. CPC A61M 5/1452; F04B 17/00; F16H 2025/2071

(57) ABSTRACT

Disclosed herein are various embodiments of clutching mechanisms applicable to a dual reservoir pumping mechanism for a drug delivery device, the clutching mechanisms serving to allow translation of one of the reservoirs during the filling process and thereafter connecting the reservoirs together such that one can be driven relative to the other one by means of a leadscrew or other driving mechanism.

### RELATED APPLICATIONS

This application claims the benefit of U.S. Provisional Patent Application No. 63/375,986, filed Sep. 16, 2022, the entire contents of which are incorporated herein by reference in its entirety.

### BACKGROUND

Many conventional automatic drug delivery (ADD) systems are well known, including, for example, wearable drug delivery devices. The drug delivery device can be designed to deliver any type of liquid drug to a user. In specific embodiments, the drug delivery device can be, for example, an OmniPod® drug delivery device manufactured by Insulet Corporation of Acton, Massachusetts. The drug delivery device can be a drug delivery device such as those described in U.S. Pat. Nos. 7,303,549, 7,137,964, or U.S. Pat. No. 6,740,059, each of which is incorporated herein by reference in its entirety.

Such drug delivery devices typically include a positive displacement pumping mechanism. Typically, the pumping mechanism comprises a reservoir that stores the liquid drug. The liquid drug stored in the reservoir may be delivered to the user by expelling the drug from a reservoir using a driven plunger that longitudinally translates through the reservoir to force the liquid drug through a fluid port defined in the reservoir. The plunger may be longitudinally translated through the reservoir by, for example, a rigidly coupled leadscrew which pushes the plunger forward during pumping. When the reservoir is filled, the leadscrew travels backwards with the plunger. The leadscrew extends past the back of the plunger a distance equal to the stroke of the plunger plus an additional amount to allow for engagement with the drive mechanism. This leads to a space efficiency constraint when scaling the design. If the stroke of the plunger increases, the length of the leadscrew must increase by the same amount.

It is desirable to use space more efficiently and to allow for a variable amount of drug to be inserted into the reservoir.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In wearable, on-body devices, it is desirable to keep the pumping mechanism, as well as the overall drug delivery device, as small as possible to minimize the impact to the wearer. Additionally, because such drug delivery devices are typically powered by an on-board battery, it is desirable to minimize the power required to operate the device. To conserve space within the housing of the drug delivery device, the positive displacement pumping mechanism may use a design comprising a double reservoir configuration having a larger, outer reservoir and a smaller, inner reservoir wherein the inner reservoir has a cross-sectional shape slightly smaller than the outer reservoir such that the inner reservoir can linearly translate through the outer reservoir, acting as a plunger for the outer reservoir. The two reservoirs are in fluid communication with each other via a rigid hollow rod which is disposed between the inner and outer reservoirs and which supports a static plunger for the inner reservoir such that, as the inner reservoir is linearly translated into the outer reservoir, the inner reservoir forces a fluid from the outer reservoir, through the hollow rod and into the inner reservoir. The static plunger in the inner reservoir acts to force fluid from the inner reservoir through an outlet fluid port as the inner reservoir is linearly translated into the outer reservoir. Some examples of a double reservoir pumping mechanism are shown in U.S. Provisional Patent Application 63/304,270, filed Jan. 8, 2022, the contents of which are incorporated herein in their entirety.

In many instances, the reservoir of the drug delivery device is filled by the user and can be filled with a variable amount of insulin. After the reservoir has been filled, the reservoir must be engaged with the drive for pumping to ensue. For double reservoir configurations, which use a telescoping reservoir assembly, the drive typically sits next to the reservoir. During filling, one of the two reservoir bodies translates while the other stays stationary. To deliver the liquid drug, the two reservoir bodies must effectively move toward each other, which can be accomplished by moving either one of the reservoir bodies while the other stays stationary. During filling, however, it is desirable that the moving reservoir is not coupled to the drive mechanism so as to allow the moving reservoir to move freely, motivated by the pressure of the incoming liquid drug and unencumbered by the drive mechanism.

Disclosed herein are several alternate embodiments of mechanisms that prepare a double reservoir pumping mechanism for pumping after the reservoirs have been filled with a variable amount of a liquid drug. In exemplary embodiments, the liquid drug can be insulin, GLP-1, pramlintide, morphine or other pain medicines, blood pressure drugs, arthritis drugs, chemotherapy drugs, fertility drugs, or the like, or co-formulations of two or more of GLP-1, pramlintide, and insulin.

In some embodiments disclosed herein, a drive mechanism is coupled to the inner reservoir of the dual reservoir configuration and the outer reservoir translates during the filling process. Thereafter, the outer reservoir is fixed with respect to the housing of the drug delivery device by a clutch mechanism to prevent further movement of the outer reservoir during pumping of the liquid drug.

In other embodiments, the outer reservoir body is fixed with respect to the housing of the drug delivery device and the inner reservoir body is disengaged from the drive mechanism during the filling process to allow translation of the inner reservoir. Thereafter, the drive mechanism is coupled to the inner reservoir via a clutch mechanism.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 illustrates a functional block diagram of an exemplary system suitable for use with the devices disclosed herein.

FIGS. 2(a-b) illustrate a first aspect of the invention wherein the outer reservoir translates during the filling process and is thereafter fixed with respect to a housing of the drug delivery device by a clutch mechanism.

FIGS. 3(a-b) illustrate an embodiment of a clutch mechanism comprising a lever and a brake pad engaging the outer reservoir.

FIG. 4(a) illustrates an embodiment of a clutch mechanism comprising mating sheets of Velcro. FIG. 4(b) illustrates an embodiment of a clutch mechanism comprising mating sheets of contact-activated adhesive.

FIG. 5 illustrates an embodiment of a clutch mechanism comprising a clamp disposed around an outer surface of the outer reservoir.

FIGS. 6(a-b) illustrate embodiments of a clutch mechanism using a guy line.

FIGS. 7(a-b) illustrate embodiments of a clutch mechanism using one or more bi-stable mechanisms.

FIGS. 8(a-b) illustrate a second aspect of the invention wherein the outer reservoir is fixed to a housing of the drug delivery device and wherein the inner reservoir is disconnected from the drive mechanism during the filling process so as to allow translation of the inner reservoir and thereafter wherein the inner reservoir is coupled to the drive mechanism via a clutch mechanism. The clutch mechanism herein comprises a torsional spring forcing the tube nut into threaded engagement with the leadscrew.

FIGS. 9(a-b) illustrate an embodiment of the clutch mechanism utilizing a spring-driven collet.

FIGS. 10(a-c) illustrate an embodiment of the clutch mechanism utilizing a frictional engagement between the tube nut and the leadscrew.

FIG. 11 illustrates an embodiment of clutch mechanism utilizing a leadscrew having a longitudinal, unthreaded notch defined thereon.

FIGS. 12(a-b) illustrate an embodiment of a clutch mechanism utilizing a clamp coupled to the inner reservoir for engaging the leadscrew.

FIGS. 13(a-b) illustrate an embodiment of a clutch mechanism utilizing a spring-driven wedge for engaging the leadscrew.

FIGS. 14(a-c) illustrate an embodiment of a clutch mechanism utilizing a spring-driven clamp for engaging the leadscrew.

FIG. 15 illustrates an embodiment of a clutch mechanism utilizing a torsional spring for engaging the leadscrew.

FIGS. 16(a-c) illustrate an embodiment of a clutch mechanism utilizing a spring-driven clamp disposed on an outer surface of the tube nut and coupled to the inner reservoir.

FIGS. 17(a-b) illustrate a second embodiment of a clutch mechanism utilizing a spring-driven clamp disposed on an outer surface of the tube nut and coupled to the inner reservoir.

FIGS. 18(a-c) illustrate an embodiment of a clutch mechanism utilizing a spring driven clamp coupled to a tube nut which engages a rod coupled to the inner reservoir.

FIGS. 19(a-b) illustrate an embodiment of a clutch mechanism utilizing a spring driven collet coupled to the tube nut which engages a rod coupled to the inner reservoir.

FIGS. 20(a-b) illustrate an embodiment of a clutch mechanism utilizing a two-bodied torsional spring coupled to a rod portion of the inner reservoir and to the tube nut.

### DETAILED DESCRIPTION

This disclosure presents various systems, components, and methods for moving a liquid drug from a liquid reservoir in a drug delivery device to a patient interface, such as a needle or cannula. The embodiments described herein provide one or more advantages over conventional, prior art systems, components, and methods, namely, a smaller overall footprint of the drug delivery device.

Various embodiments of the present invention include systems and methods for delivering a medication to a user using a drug delivery device, either autonomously, or in accordance with a wireless signal received from an electronic device. In various embodiments, the electronic device may be a user device comprising a smartphone, a smart watch, a smart necklace, a module attached to the drug delivery device, or any other type or sort of electronic device that may be carried by the user or worn on the body of the user and that executes an algorithm that computes the times and dosages of delivery of the medication.

For example, the user device may execute an “artificial pancreas” (AP) algorithm that computes the times and dosages of delivery of insulin. The user device may also be in communication with a sensor, such as a glucose sensor or a continuous glucose monitor (CGM), that collects data on a physical attribute or condition of the user, such as a glucose level. The sensor may be disposed in or on the body of the user and may be part of the drug delivery device or may be a separate device.

Alternatively, the drug delivery device may be in communication with the sensor in lieu of or in addition to the communication between the sensor and the user device. The communication may be direct (if, e.g., the sensor is integrated with or otherwise a part of the drug delivery device) or remote/wireless (if, e.g., the sensor is disposed in a different housing than the drug delivery device). In these embodiments, the drug delivery device contains computing hardware (e.g., a processor, memory, firmware, etc.) that executes some or all of the algorithm that computes the times and dosages of delivery of the medication.

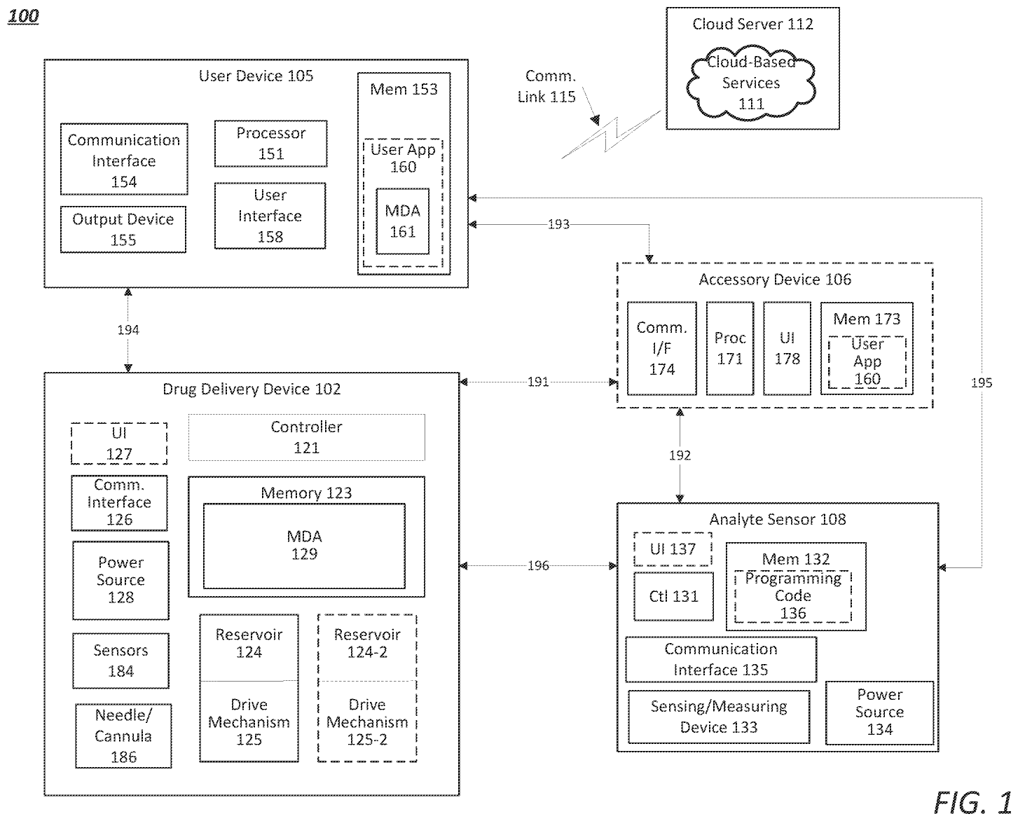

FIG. 1 illustrates a functional block diagram of an exemplary drug delivery system 100 suitable for implementing the systems and methods described herein. The drug delivery system 100 may implement (and/or provide functionality for) a medication delivery algorithm, such as an artificial pancreas (AP) application, to govern or control the automated delivery of a drug or medication, such as insulin, to a user (e.g., to maintain euglycemia—a normal level of glucose in the blood). The drug delivery system 100 may be an automated drug delivery system that may include a drug delivery device 102 (which may be wearable), an analyte sensor 108 (which may also be wearable), and a user device 105.

Drug delivery system 100, in an optional example, may also include an accessory device 106, such as a smartwatch, a personal assistant device, a smart insulin pen, or the like, which may communicate with the other components of system 100 via either a wired or wireless communication links 191-193.

### User Device

The user device 105 may be a computing device such as a smartphone, a smartwatch, a tablet, a personal diabetes management (PDM) device, a dedicated diabetes therapy management device, or the like. In an example, user device 105 may include a processor 151, device memory 153, a user interface 158, and a communication interface 154. The user device 105 may also contain analog and/or digital circuitry that may be implemented as a processor 151 for executing processes based on programming code stored in device memory 153, such as user application 160 incorporating medication delivery algorithm (MDA) 161 to manage a user's blood glucose levels and for controlling the delivery of the drug, medication, or therapeutic agent to the user, as well for providing other functions, such as calculating carbohydrate-compensation dosage, a correction bolus dosage and the like as discussed below. The user device 105 may be used to activate, deactivate, trigger a needle/canula insertion, program, adjust settings, and/or control operation of drug delivery device 102 and/or the analyte sensor 103 as well as the optional smart accessory device 106.

The processor 151 may also be configured to execute programming code stored in device memory 153, such as the user app 160. The user app 160 may be a computer application that is operable to deliver a drug based on information received from the analyte sensor 103, the cloud-based services 111 and/or the user device 105 or optional accessory device 106. The memory 153 may also store programming code to, for example, operate the user interface 158 (e.g., a touchscreen device, a camera, or the like), the communication interface 154 and the like. The processor 151, when executing user app 160, may be configured to implement indications and notifications related to meal ingestion, blood glucose measurements, and the like. The user interface 158 may be under the control of the processor 151 and be configured to present a graphical user interface that enables the input of a meal announcement, adjust setting selections and the like as described herein.

In a specific example, when the user app 160 includes MDA 161, the processor 151 is also configured to execute a diabetes treatment plan (which may be stored in a memory) that is managed by user app 160. In addition to the functions mentioned above, when user app 160 is an AP application, it may further provide functionality to determine a carbohydrate-compensation dosage, a correction bolus dosage and determine a real-time basal dosage according to a diabetes treatment plan. In addition, as an MDA 161, user app 160 provides functionality to output signals to the drug delivery device 102 via communications interface 154 to deliver the determined bolus and/or basal dosages.

The communication interface 154 may include one or more transceivers that operate according to one or more radio-frequency protocols. In one embodiment, the transceivers may comprise a cellular transceiver and a Bluetooth® transceiver. The communication interface 154 may be configured to receive and transmit signals containing information usable by user app 160.

User device 105 may be further provided with one or more output devices 155 which may be, for example, a speaker or a vibration transducer, to provide various signals to the user.

### Drug Delivery Device

In various exemplary embodiments, drug delivery device 102 may include a reservoir 124 and drive mechanism 125, which are controllable by controller 121, executing a medication delivery algorithm (MDA) 129 stored in memory 123, which may perform some or all of the functions of the AP application described above, such that user device 105 may be unnecessary for drug delivery device 102 to carry out drug delivery and control. Alternatively, controller 121 may act to control reservoir 124 and drive mechanism 125 based on signals received from user app 160 executing on a user device 105 and communicated to drug delivery device 102 via communication link 194. Drive mechanism 125 may operate to longitudinally translate a plunger through the reservoir, so as to force the liquid drug through an outlet fluid port to needle/cannula 186. Alternatively, other types of drive mechanisms may be used.

Reservoir 124 may comprise a double reservoir, as depicted in subsequent figures, where two bodies can each contain drug and can move with respect to each other. In an alternate embodiment, drug delivery device 102 may also include an optional second or additional reservoir 124-2 and second drive mechanism 125-2 which enables the independent delivery of two different liquid drugs. Reservoir 124-2 may similarly be a double reservoir as depicted in subsequent figures. As an example, reservoir 124 may be filled with insulin, while reservoir 124-2 may be filled with glucagon, or pramlintide, or GLP-1. In some embodiments, each of reservoirs 124, 124-2 may be configured with a separate drive mechanism 125, 125-2, respectively, which may be separately controllable by controller 121 under the direction of MDA 129. Both reservoirs 124, 124-2 may be connected to a common needle/cannula 186.

Drug delivery device 102 may be optionally configured with a user interface 127 providing a means for receiving input from the user and a means for outputting information to the user. User interface 127 may include, for example, light-emitting diodes, buttons on a housing of drug delivery device 102, a sound transducer, a micro-display, a microphone, an accelerometer for detecting motions of the device or user gestures (e.g., tapping on a housing of the device) or any other type of interface device that is configured to allow a user to enter information and/or allow drug delivery device 102 to output information for presentation to the user (e.g., alarm signals or the like).

Drug delivery device 102 includes a patient interface 186 for interfacing with the user to deliver the liquid drug. Patient interface may be, for example, a needle or cannula for delivering the drug into the body of the user (which may be done subcutaneously, intraperitoneally, or intravenously). Drug delivery device 102 may further include a mechanism for inserting the needle/cannula 186 into the body of the user, which may be integral with or attachable to drug delivery device 102. The insertion mechanism may comprise, in one embodiment, an actuator that inserts the needle/cannula 186 under the skin of the user and thereafter retracts the needle, leaving the cannula in place. The actuator may be triggered by user device 105 or may be a manual firing mechanism comprising springs or other energy storing mechanism, which causes the needle/cannula 186 to penetrate the skin of the user.

In one embodiment, drug delivery device 102 includes a communication interface 126, which may be a transceiver that operates according to one or more radio-frequency protocols, such as Bluetooth®, Wi-Fi, near-field communication, cellular, or the like. The controller 121 may, for example, communicate with user device 105 and an analyte sensor 108 via the communication interface 126.

In some embodiments, drug delivery device 102 may be provided with one or more sensors 184. The sensors 184 may include one or more of a pressure sensor, a power sensor, or the like that are communicatively coupled to the controller 121 and provide various signals. For example, a pressure sensor may be configured to provide an indication of the fluid pressure detected in a fluid pathway between the patient interface 186 and reservoir 124. The pressure sensor may be coupled to or integral with the actuator for inserting the patient interface 186 into the user. In an example, the controller 121 may be operable to determine a rate of drug infusion based on the indication of the fluid pressure. The rate of drug infusion may be compared to an infusion rate threshold, and the comparison result may be usable in determining an amount of insulin onboard (IOB) or a total daily insulin (TDI) amount. In one embodiment, analyte sensor 108 may be integral with drug delivery device 102.

Drug delivery device 102 further includes a power source 128, such as a battery, a piezoelectric device, an energy harvesting device, or the like, for supplying electrical power to controller 121, memory 123, drive mechanisms 125 and/or other components of drug delivery device 102.

Drug delivery device 102 may be configured to perform and execute processes required to deliver doses of the medication to the user without input from the user device 105 or the optional accessory device 106. As explained in more detail, MDA 129 may be operable, for example, to determine an amount of insulin to be delivered, JOB, insulin remaining, and the like and to cause controller 121 to activate drive mechanism 125 to deliver the medication from reservoir 124. MDA 129 may take as input data received from the analyte sensor 108 or from user app 160.

The reservoirs 124, 124-2 may be configured to store drugs, medications, or therapeutic agents suitable for automated delivery, such as those mentioned above.

Drug delivery device 102 may be a wearable device and may be attached to the body of a user, such as a patient or diabetic, at an attachment location and may deliver any therapeutic agent, including any drug or medicine, such as insulin or the like, to a user at or around the attachment location. A surface of drug delivery device 102 may include an adhesive to facilitate attachment to the skin of a user.

When configured to communicate with an external device, such as the user device 105 or the analyte sensor 108, drug delivery device 102 may receive signals over the wired or wireless link 194 from the user device 105 or from the analyte sensor 108. The controller 121 of drug delivery device 102 may receive and process the signals from the respective external devices as well as implementing delivery of a drug to the user according to a diabetes treatment plan or other drug delivery regimen.

### Accessory Device

Optional accessory device 106 may be, a wearable smart device, for example, a smart watch (e.g., an Apple Watch®), smart eyeglasses, smart jewelry, a global positioning system-enabled wearable, a wearable fitness device, smart clothing, or the like. Accessory device 106 may alternatively be a smart insulin pen that works with drug delivery device 102 in managing blood glucose and treating diabetes of a user. Similar to user device 105, the accessory device 106 may also be configured to perform various functions including controlling or communicating with drug delivery device 102. For example, the accessory device 106 may include a communication interface 174, a processor 171, a user interface 178 and a memory 173. The user interface 178 may be a graphical user interface presented on a touchscreen display of the smart accessory device 107. The memory 173 may store programming code to operate different functions of the smart accessory device 107 as well as an instance of the user app 160, or a pared-down version of user app 160 with reduced functionality. In some instances, accessory device 107 may also include sensors of various types.

### Analyte Sensor

The analyte sensor 108 may include a controller 131, a memory 132, a sensing/measuring device 133, an optional user interface 137, a power source/energy harvesting circuitry 134, and a communication interface 135. The analyte sensor 108 may be communicatively coupled to the processor 151 of the management device 105 or controller 121 of drug delivery device 102. The memory 132 may be configured to store information and programming code 136.

The analyte sensor 108 may be configured to detect one or multiple different analytes, such as glucose, lactate, ketones, uric acid, sodium, potassium, alcohol levels or the like, and output results of the detections, such as measurement values or the like. The analyte sensor 108 may, in an exemplary embodiment, be configured as a continuous glucose monitor (CGM) to measure a blood glucose values at a predetermined time interval, such as every 5 minutes, every 1 minute, or the like. The communication interface 135 of analyte sensor 108 may have circuitry that operates as a transceiver for communicating the measured blood glucose values to the user device 105 over a wireless link 195 or with drug delivery device 102 over the wireless communication link 108. While referred to herein as an analyte sensor 108, the sensing/measuring device 133 of the analyte sensor 108 may include one or more additional sensing elements, such as a glucose measurement element, a heart rate monitor, a pressure sensor, or the like. The controller 131 may include discrete, specialized logic and/or components, an application-specific integrated circuit, a microcontroller, or processor that executes software instructions, firmware, programming instructions stored in memory (such as memory 132), or any combination thereof.

Similar to the controller 121 of drug delivery device 102, the controller 131 of the analyte sensor 108 may be operable to perform many functions. For example, the controller 131 may be configured by programming code 136 to manage the collection and analysis of data detected by the sensing and measuring device 133.

Although the analyte sensor 108 is depicted in FIG. 1 as separate from drug delivery device 102, in various embodiments, the analyte sensor 108 and drug delivery device 102 may be incorporated into the same unit. That is, in various examples, the analyte sensor 108 may be a part of and/or integral with drug delivery device 102 and contained within the same housing as drug delivery device 102 or an attachable housing thereto. In such an example configuration, the controller 121 may be able to implement the functions required for the proper delivery of the medication alone without any external inputs from user device 105, the cloud-based services 111, another sensor (not shown), the optional accessory device 106, or the like.

### Cloud-Based Services

Drug delivery system 100 may communicate with or receive services from a cloud server 122 providing cloud-based services 111. Services provided by cloud server 112 may include data storage that stores personal or anonymized data, such as blood glucose measurement values, historical IOB or TDI, prior carbohydrate-compensation dosage, and other forms of data. In addition, the cloud-based services 111 may process anonymized data from multiple users to provide generalized information related to TDI, insulin sensitivity, IOB and the like. The communication link 115 that couples the cloud server 112 to other components of system 100, for example, devices 102, 105, 106, 108 of system 100 may be a cellular link, a Wi-Fi link, a Bluetooth® link, or a combination thereof.

### Communication Links

The wireless communication links 115 and 191-196 may be any type of wireless link operating using known wireless communication standards or proprietary standards. As an example, the wireless communication links 191-196 may provide communication links based on Bluetooth®, Zigbee®, Wi-Fi, a near-field communication standard, a cellular standard, or any other wireless protocol via the respective communication interfaces 126, 135, 154 and 174.

### Operational Example

In an operational example, user application 160 implements a graphical user interface that is the primary interface with the user and is used to activate drug delivery device 102, trigger a needle/cannula insertion, start and stop drug delivery device 102, program basal and bolus calculator settings for manual mode as well as program settings specific for automated mode (hybrid closed-loop or closed-loop).

User app 160, provides a graphical user interface 158 that allows for the use of large text, graphics, and on-screen instructions to prompt the user through the set-up processes and the use of system 100. It may also be used to program the user's custom basal insulin delivery profile, accept a recommended basal insulin delivery profile, check the status of drug delivery device 102, initiate bolus doses of insulin, make changes to a patient's insulin delivery profile, handle system alerts and alarms, or allow the user to switch between automated mode and manual mode.

User app 160 may be configured to operate in a manual mode in which user app 160 will deliver insulin at programmed basal rates and user-defined bolus amounts with the option to set temporary basal profiles. The controller 121 will also have the ability to function as a sensor-augmented pump in manual mode, using sensor glucose data provided by the analyte sensor 108 to populate the bolus calculator.

User app 160 may be configured to operate in an automated mode in which user app 160 supports the use of one or multiple target blood glucose values that may be adjusted manually or automatically by the system. For example, in one embodiment, target blood glucose values can range from 110-150 mg/dL, in 10 mg/dL increments, in 5 mg/dL increments, or other increments, but preferably 10 mg/dL increments. The experience for the user will reflect current setup flows whereby the healthcare provider assists the user to program basal rates, glucose targets and bolus calculator settings. These in turn will inform the user app 160 for insulin dosing parameters. The insulin dosing parameters will be adapted over time based on the total daily insulin (TDI) delivered during each use of drug delivery device 102. A temporary hypoglycemia protection mode or an activity mode may be implemented by the user for various time durations in automated mode. With a hypoglycemia protection mode or an activity mode, the algorithm reduces insulin delivery and is intended for use over temporary durations when insulin sensitivity is expected to be higher, such as during exercise or fasting.

The user app 160 (or MDA 129) may provide periodic insulin micro-boluses based upon past glucose measurements and/or a predicted glucose over a prediction horizon (e.g., 60 minutes). Optimal post-prandial control may require the user to give meal boluses in the same manner as current pump therapy, but normal operation of the user app 160 will compensate for missed meal boluses and mitigate prolonged hyperglycemia. The user app 160 uses a control-to-target strategy that attempts to achieve and maintain a set target glucose value, thereby reducing the duration of prolonged hyperglycemia and hypoglycemia.

In some embodiments, user device 105 and the analyte sensor 108 may not communicate directly with one another. Instead, data (e.g., blood glucose readings) from analyte sensor may be communicated to drug delivery device 102 via link 196 and then relayed to user device 105 via link 194. In some embodiments, to enable communication between analyte sensor 108 and user device 105, the serial number of the analyte sensor must be entered into user app 160.

User app 160 may provide the ability to calculate a suggested bolus dose through the use of a bolus calculator. The bolus calculator is provided as a convenience to the user to aid in determining the suggested bolus dose based on ingested carbohydrates, most-recent blood glucose readings (or a blood glucose reading if using fingerstick), programmable correction factor, insulin to carbohydrate ratio, target glucose value, and insulin on board (IOB). IOB is estimated by user app 160 taking into account any manual bolus and insulin delivered by the algorithm.

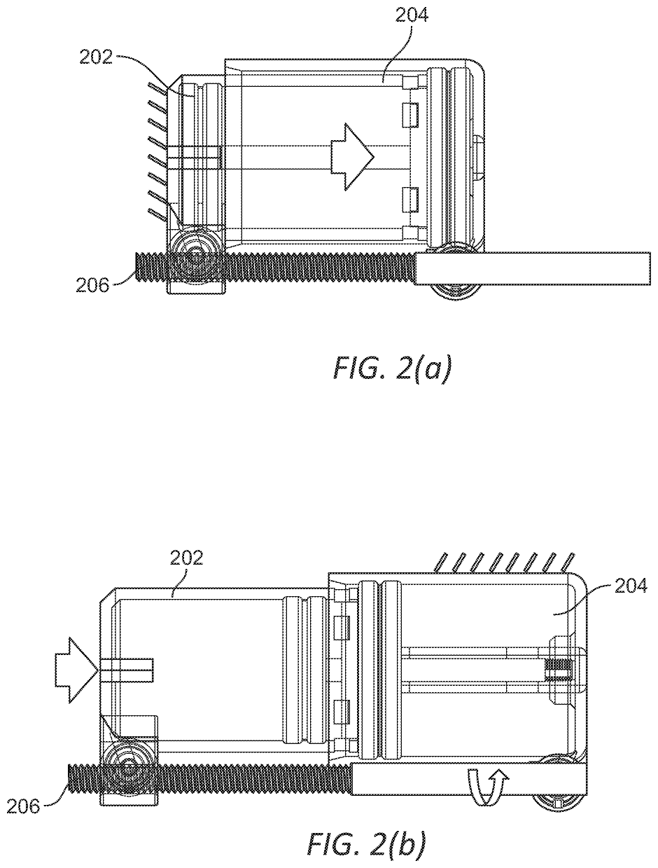

In a first aspect of the invention, during the filling process, the inner reservoir 202 is held stationary with respect to the housing of the drug delivery device 102 and the outer reservoir 204 is free to move during the filling process. This is illustrated in FIG. 2(a). Various mechanisms may be used to hold the inner reservoir 202 stationary during the filling process. In the embodiment shown in FIG. 2(a) leadscrew 206 holds inner reservoir 202 stationary during the filling process. A clutch mechanism (not shown), disconnects outer reservoir 204 from the housing of drug delivery device 102, allowing outer reservoir 204 to translate motivated (i.e., driven) by the pressure of the liquid drug as it enters reservoirs 202, 204. After completion of the filling process, the clutch mechanism engages the outer reservoir 204, thereby rendering it stationary with respect to the housing of the drug delivery device 102, allowing the drive mechanism to move the inner reservoir 202 to pump the liquid drug. This is illustrated in FIG. 2(b). Various embodiments of the clutch mechanism, which will now be explained and illustrated, are directed to this aspect of the invention. In various embodiments, it will be appreciated that either the outer reservoir or the inner reservoir can move with respect to the other, fixed, reservoir.

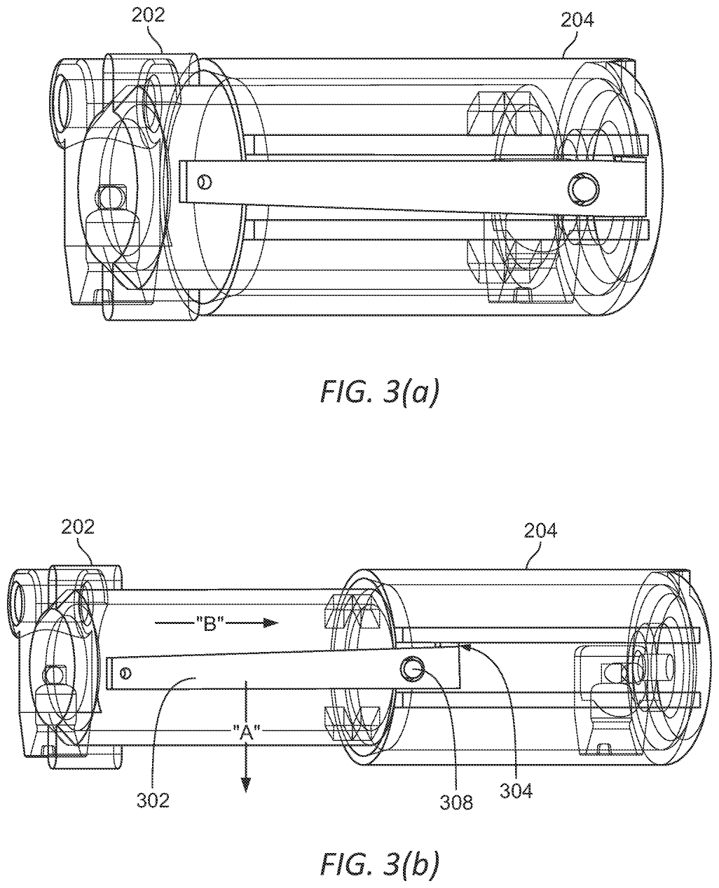

FIGS. 3(a-b) show a first embodiment of the first aspect of the invention. In this embodiment, outer reservoir 204 is provided with a brake mechanism comprising lever 302 and brake pad 304. Before and during the filling process, shown in FIG. 3(a), the brake mechanism is disengaged, allowing outer reservoir 204 (or inner reservoir 202) to move freely while inner reservoir 202 (or outer reservoir 204) is held stationary by the drive mechanism (not shown in this figure). After the filling process is complete, shown in FIG. 3(b), the brake mechanism is engaged by moving lever 302 in direction “A” so as to pivot lever 302 around pivot point 308. The pivoting action brings brake pad 304 into contact with an outer surface of outer reservoir 204 thereby rendering it stationary with respect to the housing of the drug delivery device 102. During the pumping of the liquid drug, inner reservoir 202 moves in direction “B” with respect to outer reservoir 204, motivated by the drive mechanism, to cause pumping of the liquid drug.

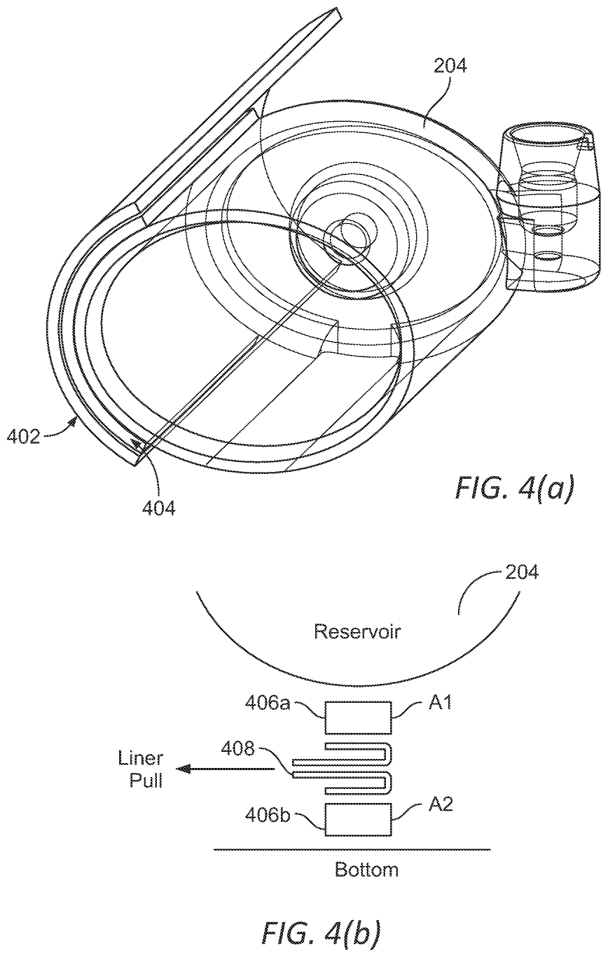

In a second embodiment, shown in FIG. 4(a), outer reservoir 204 is configured with (or comprises) a first sheet of hook and loop fasteners 404 or other fastener material, which may be adhesively attached to an outer surface of outer reservoir 204. A second, mating sheet of hook and loop fasteners 402 is adhesively attached to the housing of drug delivery device 102. During the filling process, a sheath (not shown) is disposed between hook and loop fastener sheets 402, 404 to prevent engagement therebetween. This allows reservoir 204 to move with respect to the housing of the drug delivery device 102. Inner reservoir 202 (not shown in FIG. 4(a)) is held stationary by the drive mechanism. After the filling process is complete, the sheath is removed and hook and loop fastener sheets 402, 404 engage each other to prevent further movement of outer reservoir 204. During the pumping process, inner reservoir 202 moves with respect to now-stationary outer reservoir 204, motivated by the drive mechanism. The sheath disposed between hook and loop fastener sheets 402 and 404 may be removed by any known mechanism.

In a variation of this embodiment, shown in FIG. 4(b), a two-part adhesive may be used which activates when the two parts make contact with each other. A first part of the adhesive 406a is coupled to the reservoir 202 while second part 406b is coupled to a housing of drug delivery device 102. During the filling process, liner 408 prevents contact between parts 406a and 406b of the adhesive. After the filling process is complete, the liner 408 is pulled from between parts 406a, 406b allowing the parts to make contact with each other and activates the adhesive so as to hold outer inner reservoir 204 in a stationary position with respect to the housing of drug delivery device 102. Liner 408 may be pulled from between adhesive parts 406a, 406b by any known means.

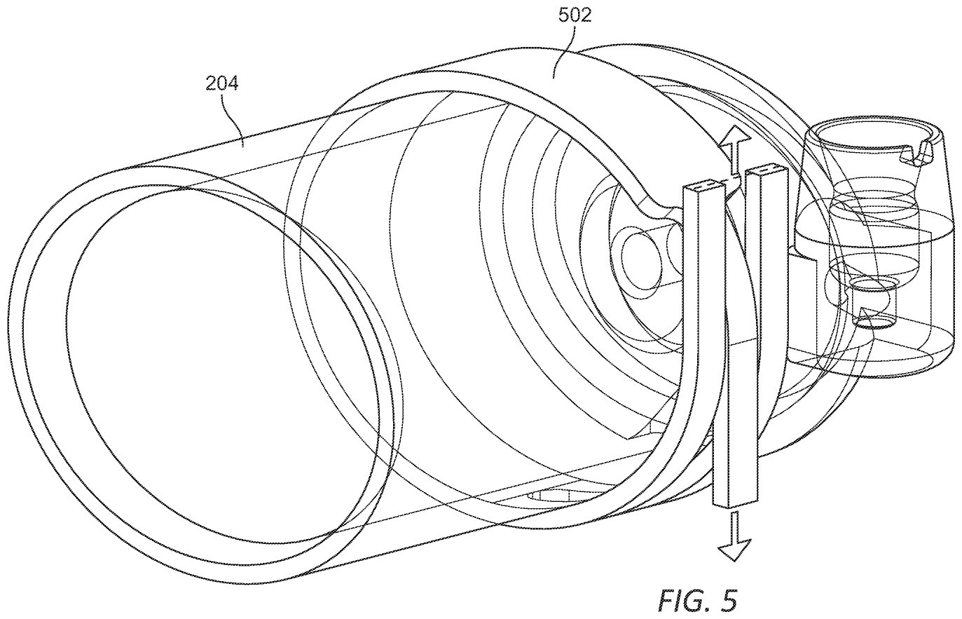

In a third embodiment, shown in FIG. 5, outer reservoir 204 is configured with a clamp 502 which is directly or indirectly coupled to a housing of drug delivery device 102. In some embodiments, the clamp 502 is ring-shaped, more specifically open ring-shaped wherein each end comprises at least one arm, in particular at least one straight arm. During the filling process, clamp 502 is tensioned by moving the arms of the clamp in a direction opposite that shown by the arrows in FIG. 5, to disengage clamp 502 from outer reservoir 204 and to allow outer reservoir 204 to translate with respect to inner reservoir 202. In some embodiments, during filling, the clamp's arms are moved opposite one another such that the inner diameter of clamp 502 is increased, in particular wherein the arms overlap, and the end of the arms are brought closer to one another. Inner reservoir 202 is held stationary by the drive mechanism. After the filling process is complete, clamp 502 is tightened around the outside circumference of outer reservoir 204 by releasing the tension in clamp 502, causing the arms of clamp 502 to move in the direction of the arrows in FIG. 5. In some embodiments, after filling, the clamp's arms are moved opposite one another such that the inner diameter of clamp 502 is decreased, in particular wherein the arms overlap, and the end of the arms are brought further from one another. Clamp 502 thus engages with outer reservoir 204 to render outer reservoir 204 stationary with respect to the housing of drug delivery device 102. During the pumping process, inner reservoir 202 moves with respect to now-stationary outer reservoir 204, motivated by the drive mechanism. Clamp 502 may move into the tensioned state by any known mechanism or trigger.

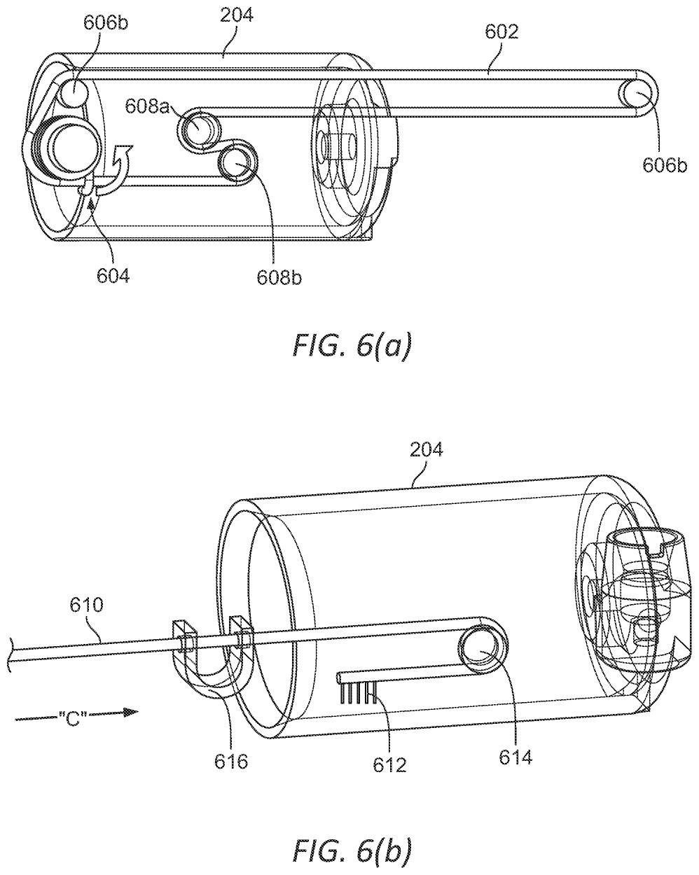

FIGS. 6(a-b) show two separate embodiments using a guy line 602. In a first embodiment, shown in FIG. 6(a), guy line 602 is coupled to the housing of drug delivery device 102 at points 606a and 606b (for example, a first point (e.g., 606a) connected to the housing and a second point (e.g., 606b) connected to the housing, wherein the outer reservoir 204 is configured to translate between the first point 606a and the second point 606b) and, in addition, is coupled to outer reservoir 204 at connection points 608 (for example, a third point 608a and a fourth point 608b connected to the outer reservoir 204, wherein the fourth point 608b is disposed closer to the second point 606b as compared to the third point 608a, and in particular wherein the third point 608a and fourth point 608b are offset relative to one another in a direction perpendicular to the direction of translation of the outer reservoir 204). Guy line 602 is able to pivot about (i.e., move along) connection points 606a, 606b, 608a and 608b. During the filling process, slack in guy line 602 allows for the outer reservoir 204 to move with respect inner reservoir 202 (not shown in this example). At the completion of the filling process, spring 604 is released and tensions guy line 602 preventing further movement of outer reservoir 204 with respect to the housing of drug delivery device 102.

FIG. 6(b) shows a second embodiment using a guy line 610. In this embodiment, guy line 610 is rigidly coupled to outer reservoir 204 at connection point 612 and is able to move freely around pivot point 614. The connection point 612 and/or the pivot point 614 may be connected to the outer reservoir 204. During the filling process, guy line 610 is pulled in direction “C” by the movement of reservoir in direction “C”. During the filling process, spring 616 is tensioned so as to allow the free movement of guy line 610 through holes defined in spring 616. After the filling process is complete, the tension in spring 616 is released, thereby preventing further movement of guy line 610 to render outer reservoir 204 stationary with respect to the housing of the drug delivery device 102. In particular, the through holes defined in spring 616 may align when the spring 616 is tensioned and may not align when tension in spring 616 is released. The end of guy line 610 may be stored by any known means and spooled out as outer reservoir 204 moves in direction “C”.

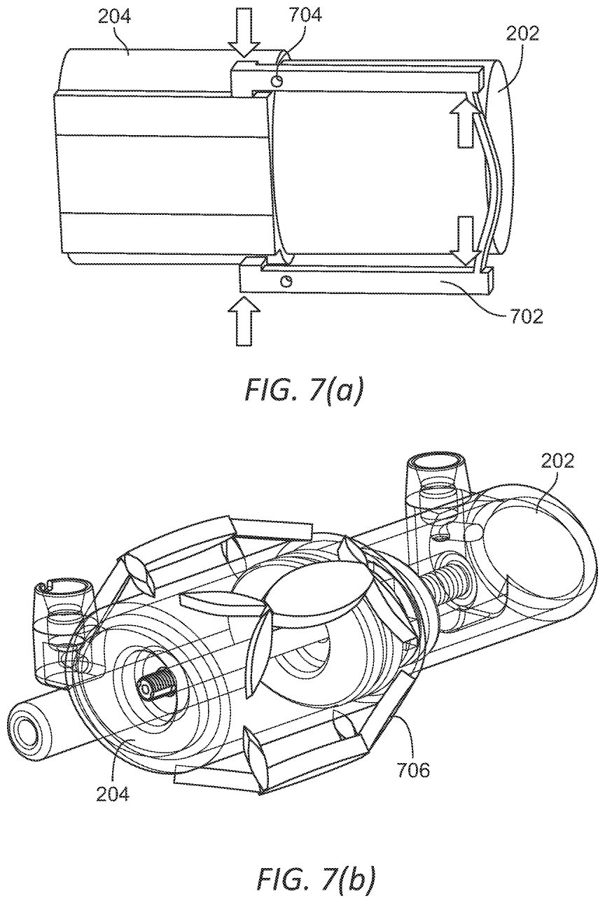

FIGS. 7(a-b) show two embodiments using bi-stable mechanisms to hold outer reservoir 204 in place after completion of the filling process. FIG. 7(a) shows a first bi-stable mechanism 702 which is coupled to a housing of the drug delivery device 102. During the filling process, the arms of bi-stable mechanism 702 pivot on pivot points 704 to a first stable position, disengaging the arms of bi-stable mechanism 702 from outer reservoir 204. After the filling process, bi-stable mechanism 702 moves to a second stable state wherein the arms of the mechanism are brought into engagement with a portion of outer surface of outer reservoir 204 to prevent further movement of outer reservoir 204. The transition of bi-stable mechanism 702 from the first stable state to the second stable state may be initiated by a triggering mechanism of any design.

FIG. 7(b) shows several bi-stable mechanisms 706 of a different design. The term “bi-stable mechanism” as used may relate to a mechanism that has two (mechanically) stable geometric configurations. Bi-stable mechanisms 706 are coupled to the housing of drug delivery device 102 and, in a first stable state, are disengaged from outer reservoir 204. After the filling process, the mechanisms 706 move to a second stable state in which they are engaged with an outer surface of outer reservoir 204 to prevent further movement of outer reservoir 204. The transition of bi-stable mechanisms 706 from the first stable state to the second stable state may be initiated by a triggering mechanism of any design. In some embodiments, the bi-stable mechanisms 706 comprise one or more leg(s) and a body, wherein the one or more leg(s) moves towards the outer reservoir 204, in particular until it engages with the outer reservoir, when transitioning from the first stable state to the second stable state and wherein the one or more leg(s) moves away from the outer reservoir 204, in particular until it disengages from the outer reservoir 204. In some embodiments, the bi-stable mechanism 706 comprises a plurality of legs, in particular 3 or 4 legs, arranged around its body. In some embodiments, the one or more leg(s) flexes relative to the body when transitioning from the first stable state to the second stable state and vice versa. In some embodiments, one or more bi-stable mechanism(s) 706 is coupled to the housing of the drug delivery device 102, more specifically 2 to 4 bi-stable mechanisms 706 are coupled to the housing of the drug delivery device 102 and in particular 3 bi-stable mechanisms are coupled to the housing of the drug delivery device 102. In some embodiments, the bi-stable mechanisms 706 are coupled to the outer reservoir 204 and the one or more leg(s) moves towards the housing of the drug delivery device 102, in particular until it engages with the housing of the drug delivery device 102, when transitioning from the first stable state to the second stable state and the one or more leg(s) move away from the housing of the drug delivery device 102, in particular until it disengages from the housing of the drug delivery device 102.

In a second aspect of the invention, during the filling process, the outer reservoir 204 is held stationary with respect to the housing of the drug delivery device 102 and the inner reservoir 202 is free to move during the filling process. In these embodiments, outer reservoir 204 is typically coupled in some manner to the housing of drug delivery device 102. Inner reservoir 202 is disengaged from the drive mechanism by a clutch mechanism and is allowed to move freely, motivated by the pressure of the liquid drug as reservoirs 202, 204 are filled. After filling, the clutch mechanism re-engages inner reservoir 202 with the drive mechanism.

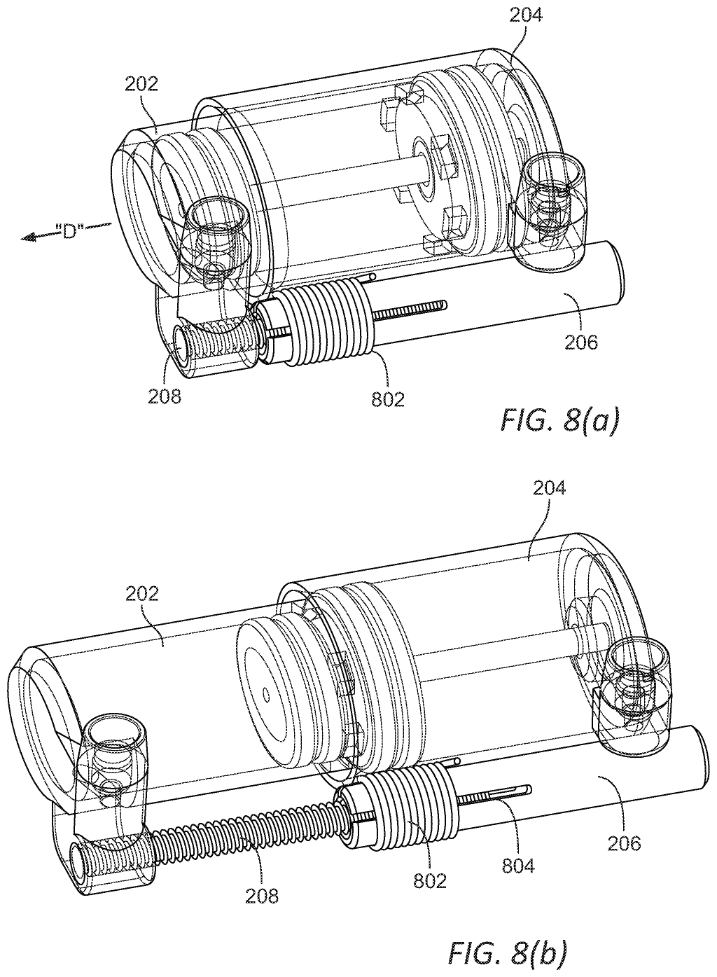

One embodiment of the second aspect of the invention is shown in FIG. 8. In this embodiment, the drive mechanism consists of tube nut 206 which is rotated to drive leadscrew 208, which is rigidly coupled to inner reservoir 202. FIG. 8(a) shows the state of the mechanism prior to filling wherein clutch 802 which, in this embodiment, comprises a torsional spring, is tensioned to disengage it from leadscrew 208. Thereby allowing leadscrew 208 to move freely within tube nut 206. In this state, leadscrew 208 is disposed within tube nut 206. As reservoirs 202, 204 are filled, inner reservoir 202 moves in direction “D” and, in the process, pulls leadscrew 208 from its previous position within tube nut 206. FIG. 8(b) shows the pumping mechanism in a filled state. Once reservoirs 202, 204 are filled, clutch mechanism 802 is un-tensioned to cause the end of tube nut 206 to close on leadscrew 208 by virtue of the squeezing of the end of tube nut 206 and the narrowing of slits 804 defined in the end of tube nut 206, forcing tube nut 206 into threaded engagement with leadscrew 208. Thereafter, rotation of tube nut 206 will cause inner reservoir 202 to move in a direction opposite direction “D” to pump the liquid drug by virtue of the threaded engagement between tube nut 206 and leadscrew 208.

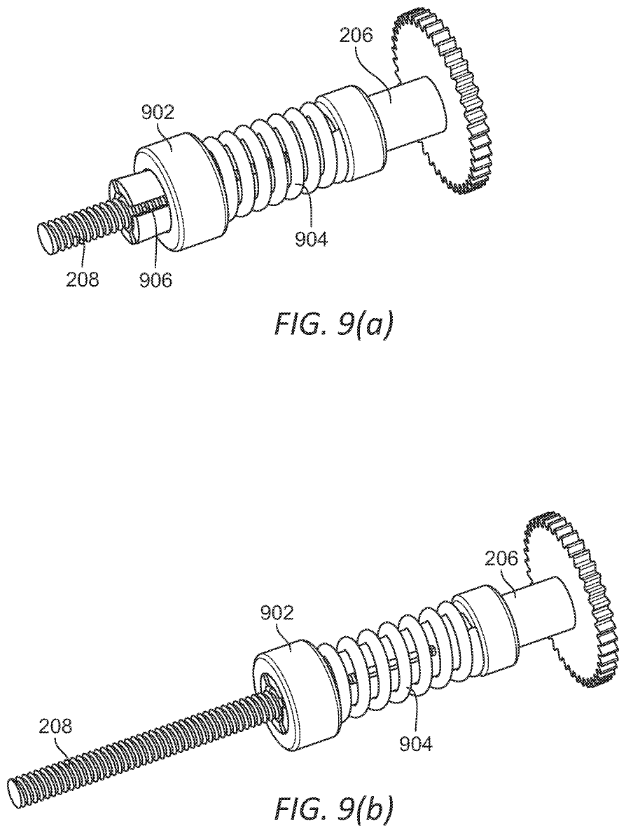

FIGS. 9(a-b) show a variation of the embodiment of FIG. 8 utilizing a different clutch mechanism. As with the embodiment of FIG. 8, during the filling process, shown in FIG. 9(a), leadscrew 208 is free to move within tube nut 206 by virtue of the spreading of slits 906 defined in the end of tube nut 206. When the filling process is complete, as shown in FIG. 9(b), the tension in spring 904 is released, thereby pushing collet 902 to the end of tube nut 206 to squeeze the end of tube nut 206 by the narrowing of slits 906 to force tube nut 206 into threaded engagement with leadscrew 208, which is coupled to outer reservoir 202. As such, rotation of tube nut 206 will cause translation of inner reservoir 206 to pump the liquid drug. Spring 904 may be held in its tensioned state, shown in FIG. 9(a), by any known means and released by a triggering mechanism of any design.

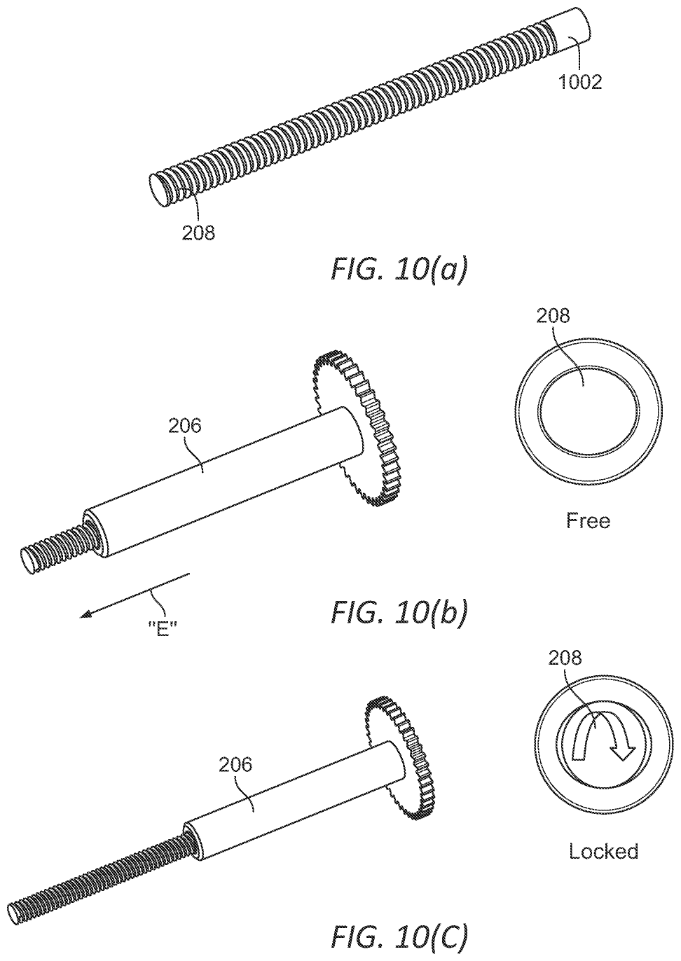

FIGS. 10(a-c) show yet another embodiment of the clutching mechanism. In this embodiment, leadscrew 206, as shown in FIG. 10(a), has a portion 1002 defined on one end thereof having an oval cross-sectional shape. Additionally, the inner diameter of tube nut 206 also has a cross-sectional shape. When the major axis of the oval portion 1002 of leadscrew 208 is aligned with the major axis of the inner diameter of tube nut 206, as shown in FIG. 10(b), leadscrew 208 is free to move within tube nut 206. During the filling process, leadscrew 208 moves in direction “E”. Once the filling process is complete, tube nut 206 rotates, for example by 90°, such that the major axis of portion 1002 of leadscrew 208 is aligned with the minor axis of the oval cross-section of the inner diameter of tube nut 206, thereby creating a frictional lock between tube nut 206 and leadscrew 208, as shown in FIG. 10(c). In this embodiment, leadscrew 208 is in threaded engagement with inner reservoir 202. Rotation of tube nut 206 also causes rotation of leadscrew 208 so as to motivate inner reservoir 202 to move in a direction opposite direction “E” by virtue of the threaded engagement between the leadscrew 208 and inner reservoir 202.

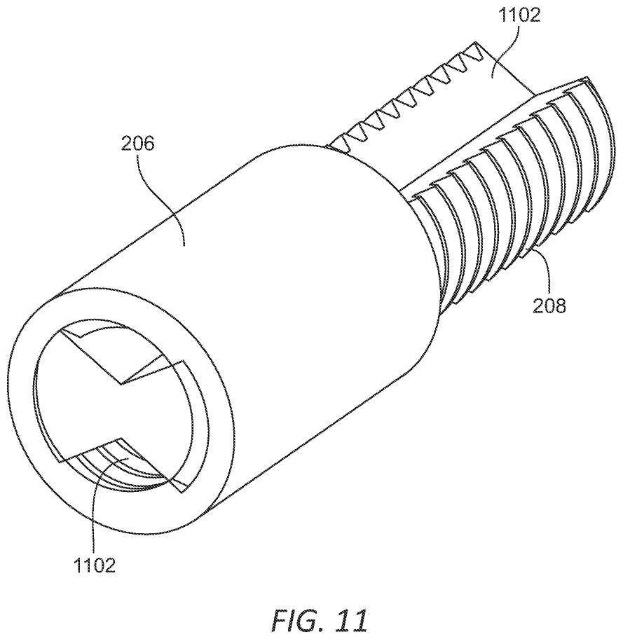

FIG. 11 shows yet another embodiment in which tube nut 206 may be positioned to allow leadscrew 208 to move freely within tube nut 206. In this arrangement, leadscrew 208 is configured with notched areas 1102 extending along a longitudinal axis of leadscrew 208. The inner diameter of tube nut 206 is configured with areas which are free from threads. When the areas having no threads on the inner diameter of tube nut 206 are aligned with the notched areas 1002 on leadscrew 208, as depicted in FIG. 11, leadscrew 208 is free to move within tube nut 206. During the filling process, these areas (having no threads) are aligned, allowing translation of inner reservoir 202, motivated by the pressure of the liquid drug as it fills reservoirs 202, 204. Thereafter, during the pumping process, tube nut 206 can be rotated to create a threaded engagement with leadscrew 208 to motivate the inner reservoir to pump the liquid drug.

One threaded side of leadscrew 208 may be larger than the other threaded side of leadscrew 208 to aid in having at least one portion of leadscrew 208 engage corresponding threads on tube nut 206 during the pumping process. Additionally, or alternatively, during the pumping process, as the leadscrew 208 (or alternatively the tube nut 206) rotates, the threads of tube nut 206 and the threads of leadscrew 208 may only engage each other part of the time. The pump driving algorithm could take this into account (viz., that part of the time, the leadscrew (or tube nut) will not rotate when the tube nut (or leadscrew) rotates), and adjust drug delivery or advancement of the delivery mechanism accordingly.

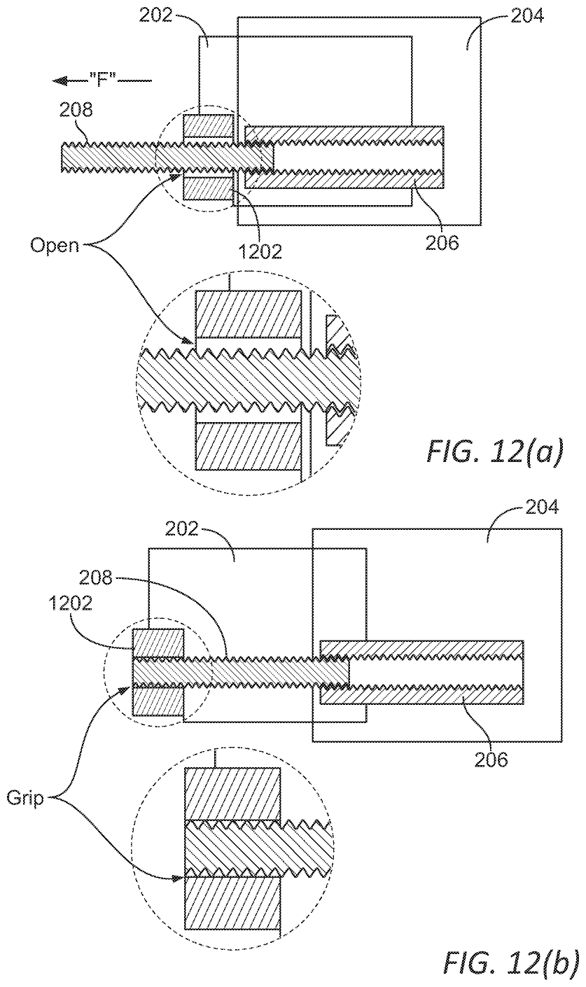

FIGS. 12(a-b) show an embodiment wherein inner reservoir 202 is configured with a clamp 1202 which engages with leadscrew 208. During the fill process, shown in FIG. 12(a), clamp 1202 is released from engagement with leadscrew 208, allowing inner reservoir 202 to move freely in direction “F”, motivated by the pressure of the liquid drug is it fills reservoirs 202, 204. When the filling process is complete, shown in FIG. 12(b), clamp 1202 is engaged with leadscrew 208 such that rotation of tube nut 206, which is in threaded engagement with leadscrew 208 motivates the translation of inner reservoir 202 in a direction opposite direction “F” to pump the liquid drug. Clamp 1202 may be engaged and disengaged from leadscrew 208 by any known means, for example, via a spring that, when tensioned releases clamp 1202 from engagement with leadscrew 208 and, when un-tensioned, forces clamp 1202 to engage leadscrew 208.

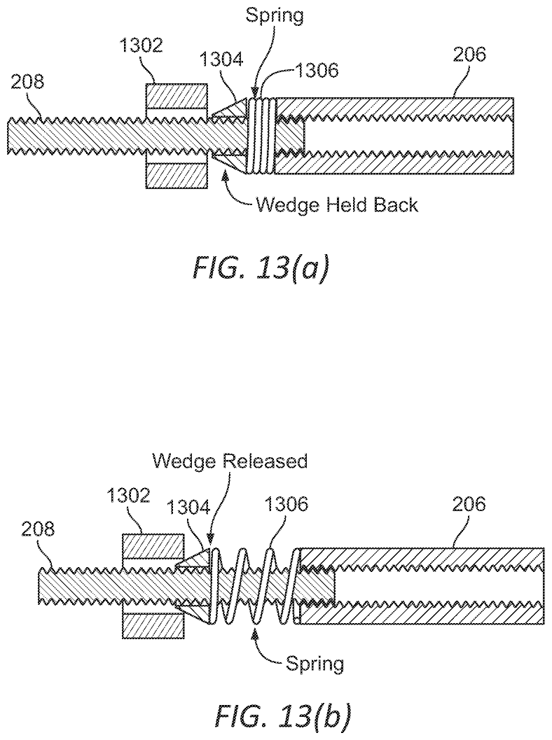

FIGS. 13(a-b) show a variation of the embodiment of FIG. 12. In this embodiment, at the completion of the fill process, shown in FIG. 13(b), a wedge 1304 is driven into engagement with a portion 1302 of inner reservoir 202, motivated by tension released in spring 1306. Wedge 1304 may be configured with slits (not visible) such that engagement of the wedge 1304 with the portion 1302 of inner reservoir 202 causes the slits in wedge 1304 to narrow, thereby clamping wedge 1304 to leadscrew 208. During the pumping process, rotation of tube nut 206 causes translation of inner reservoir 202 which is coupled to leadscrew 208 by virtue of wedge 1304. The pre-filling state is shown in FIG. 13(a) in which wedge 1304 is held back from portion 1302 and spring 1306 is tensioned. Wedge 1304 may be held back before and during the filling process by any known means and the tension in spring 1306 released via a triggering mechanism of any design.

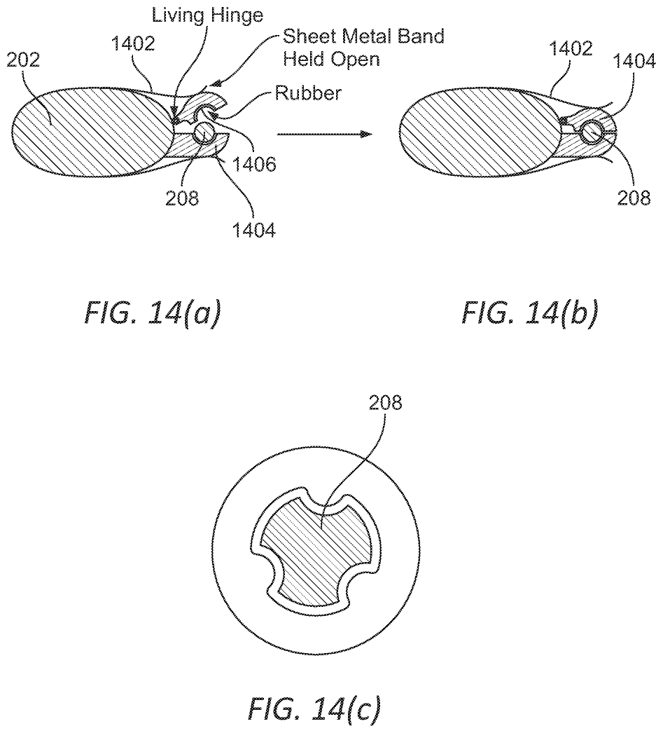

FIGS. 14(a-c) show yet another variation of a clamping mechanism. In this embodiment, inner reservoir 202 is provided with a metal band 1402 that, in the prefilled state shown in FIG. 14(a), is tensioned by being held in an open position. After the filling process is complete, the tension in metal band 1402 is released causing hinge 1404 to close around leadscrew 208 thereby coupling inner reservoir 202 to leadscrew 208, as shown in FIG. 14(b). In certain embodiments, a portion of leadscrew 208 may have a scalloped cross-sectional shape, as shown in FIG. 14(c), to facilitate a frictional engagement with the inner surfaces of hinge the 1404. In addition, the inner surfaces of hinge 1404 may be configured with rubber pads 1406 to further aid in the frictional coupling of hinge 1404 with leadscrew 208. Before and during the filling process, metal band 1402 may be held open by any known means and thereafter triggered so as to close hinge 1404 by a trigger mechanism of any design.

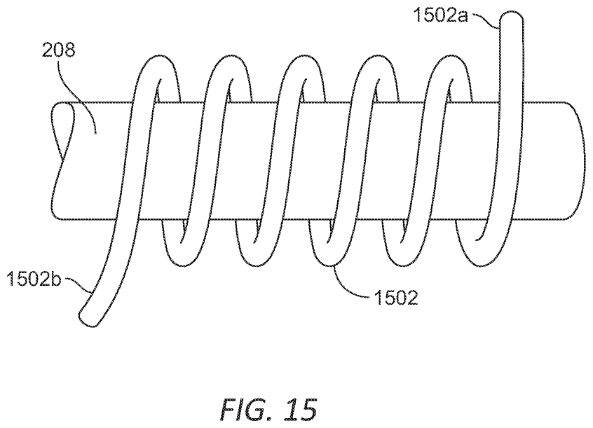

FIG. 15 shows another embodiment utilizing a clamp 1502 to couple leadscrew 208 to inner reservoir 202. In this embodiment, clamp 1502 comprises a torsional spring. During the filling process, spring 1502 is held in an open, tensioned position by movement of end 1502b. End 1502a is coupled to the inner reservoir 202. After the filling process is complete, the tension in spring 1502 is released by movement of end 1402b, causing the spring to engage leadscrew 208 to couple leadscrew 208 to the inner reservoir 202. End 1502b of spring 1502 may be manipulated by any known means and may be triggered by a triggering mechanism of any design.

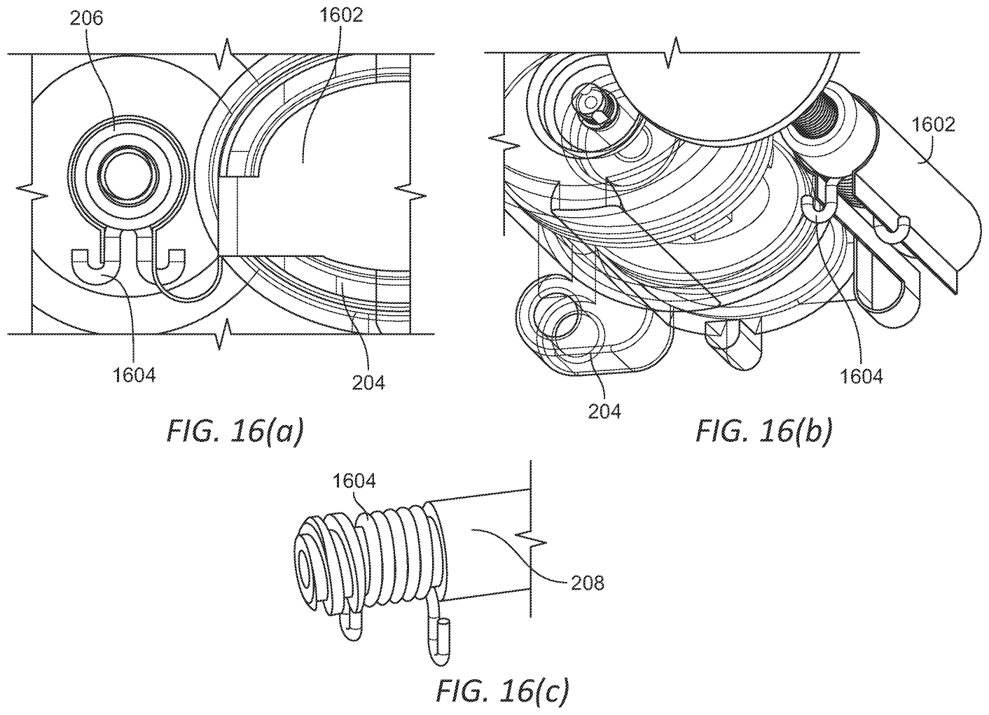

FIGS. 16(a-c) show another embodiment utilizing a clamp 1602. In this embodiment, as reservoirs 202, 204 are filled, clamp 1602, which is coupled to inner reservoir 202, is free to move over the outside surface of tube nut 206 as inner reservoir 202 translates away from outer reservoir 204. Spring 1604, which is wrapped around leadscrew 208, as shown in FIG. 16(c), is tensioned by pushing hooks on the ends of spring 1604 in opposite directions. Once the filling process is complete, the tension on spring 1604 is released and the hooks engage clamp 1602 so as to cause clamp 1602 to engage tube nut 206, thereby preventing further movement of tube nut 206 with respect to clamp 1602 and coupling tube nut 206 to the inner reservoir 202. Spring 1604 may be held in the tensioned state by any known means and released by a trigger mechanism of any design.

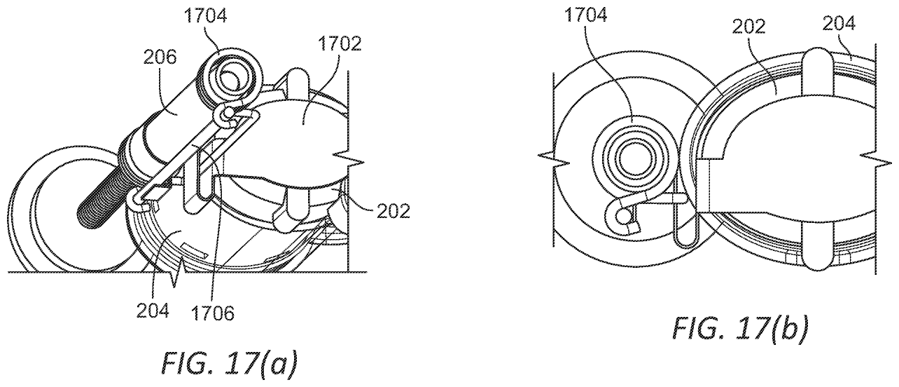

FIGS. 17(a-b) show a variation of the embodiment shown in FIGS. 16(a-c) utilizing a spring 1704 which engages a rod 1706 to push clamp 1702 into engagement with tube nut 206. During the filling process, spring 1704 is tensioned so as to relieve the pressure on rod 1706, allowing clamp 1702 to translate linearly over tube nut 206 as reservoir 202 translates away from reservoir 204. After the filling process is complete, the tension on spring 1704 is released, pushing rod 1706 against clamp 1702, causing clamp 1702 to engage tube nut 206, thereby coupling reservoir 202 to tube nut 206. Spring 1704 may be held in the tensioned state by any known means and released by a trigger mechanism of any design.

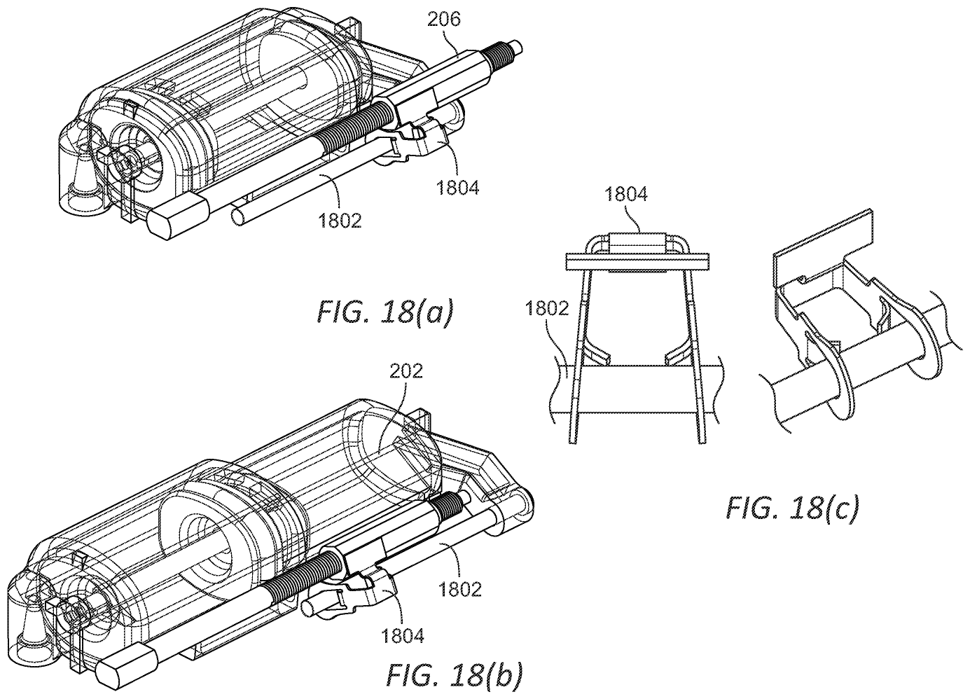

FIGS. 18(a-c) show an embodiment wherein inner reservoir 202 is coupled to a rod 1802 which, before and during the filling process, shown in FIG. 18(a), is able to translate through a clamp 1804 which is coupled to tube nut 206. Clamp 1804, shown in FIG. 18(c), is held in a tensioned state by movement of its arms toward each other to disengage them from rod 1802 and to allow rod 1802 to translate through the holes defined in the arms of clamp 1804. After the filling process is complete, shown in FIG. 18(b), the tension on the arms of clamp 1804 is released such that the arms engage rod 1802, preventing further movement of rod 1802 through the holes defined in the arms of clamp 1804, thereby coupling inner reservoir 202 to tube nut 206. The arms of clamp 1804 may be held together to tension clamp 1804 by any known means and may be released by a triggering mechanism of any design.

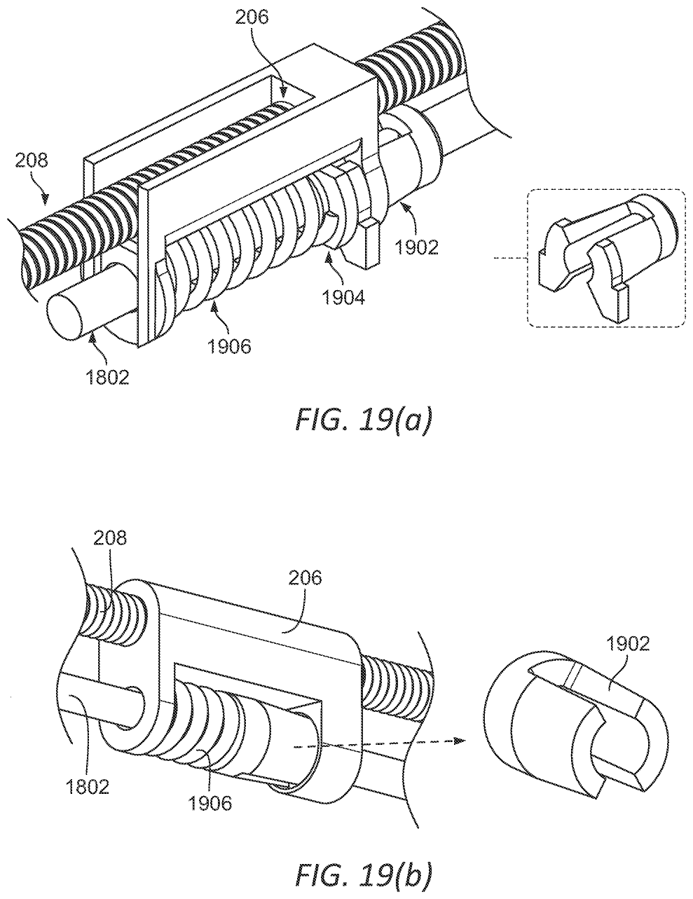

FIGS. 19(a-b) show a variation of the embodiment of FIGS. 18(a-c) in which the tube nut 206 is coupled to rod 1802 by a collet mechanism. The collet mechanism comprises spring 1906, spring plate 1904 and collet 1902. During the filling process, spring 1906 is tensioned and collet 1902 is disengaged from rod 1802, as shown in FIG. 19(b). After the filling process is complete, the tension in spring 1906 is released, thereby forcing collet 1902 through a hole defined in tube nut 206. This causes the arms of collet 1902 to compress around rod 1804, thereby coupling the tube nut 206 to rod 1802. Because rod 1802 is coupled to inner reservoir 202, as shown in FIG. 18(b), inner reservoir 202 becomes coupled to tube nut 206 when collet 1902 is engaged with rod 1802. Spring 1906 may be held in the tension state by any known means and may be released by a triggering mechanism of any design.

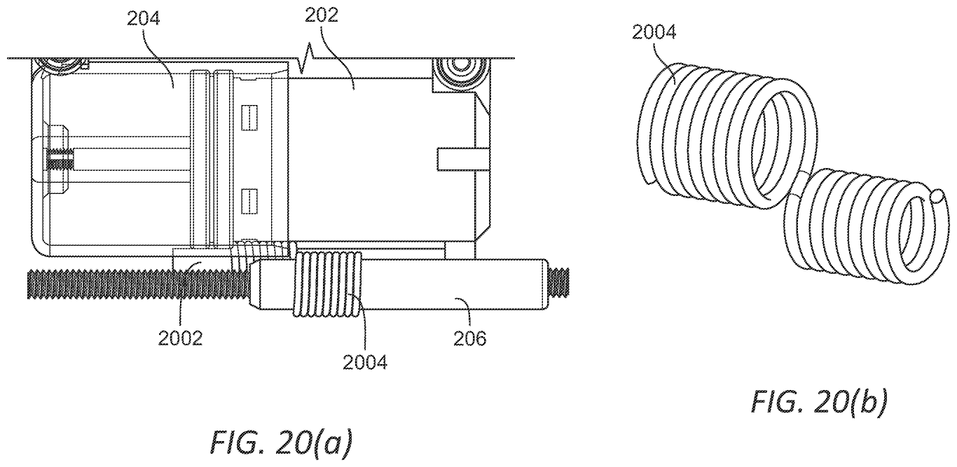

FIGS. 20(a-b) show another embodiment utilizing a two-bodied torsional spring 2004 as a clamp. Inner reservoir 202 is provided with a rod 2002 which is engaged by one body of the two-bodied torsional spring 2004. The other body of the two-bodied torsional spring 1204 is engaged with tube nut 206. During the filling process, two-bodied torsional spring 2004 is tensioned, thereby allowing rod 2002 to translate through spring 2004. After the filling process is complete, the tension on spring 2004 is released, thereby causing it to clamp on both rod 2002 and tube nut 206 so as to couple tube nut 206 to the inner reservoir 202. Spring 2004 may be held in the tensioned state by any known means and may be released by a trigger mechanism of any design.

As would be realized by one of skill in the art, many variations on the embodiments disclosed herein are possible. In particular, various sizes, materials and configurations are contemplated to be the within the scope of the invention and the invention is not meant to be limited by the specific embodiments disclosed herein. Additionally, the embodiments described are not mutually exclusive but may be used in conjunction with one another.

The following examples pertain to various embodiments disclosed herein for the needle insertion/reduction mechanism for use with an automatic drug delivery system.

Example 1 is a first embodiment of a pumping mechanism for drug delivery device comprising an outer reservoir, an inner reservoir configured to linearly translate into the outer reservoir, a drive mechanism for linearly translating the inner reservoir into the outer reservoir and a clutch mechanism for coupling the outer reservoir to a housing of the drug delivery device.

Example 2 is an extension of Example 1, or any other example disclosed herein, wherein the clutch mechanism comprises a brake pad and a lever for forcing the brake pad into engagement with an outer surface of the outer reservoir.

Example 3 is an extension of Example 1, or any other example disclosed herein, wherein the clutch mechanism comprises a first piece of Velcro attached to a housing of the drug delivery device, a second piece of mating Velcro attached to an outer surface of the outer reservoir and a sheath disposed between the first and second piece of Velcro to prevent engagement therebetween, wherein the sheath is removed after the filling of the pumping mechanism to a non-engagement of the first and second pieces of meeting Velcro to prevent further movement of the outer reservoir.

Example 4 is an extension of Example 1, or any other example disclosed herein, wherein the clutch mechanism comprises a first adhesive pad attached to a housing of the drug delivery device, a second adhesive pad attached to an outer surface of the outer reservoir and the liner disposed between the first adhesive pad and second adhesive pad wherein the liner is removed after the filling of the pumping mechanism and further wherein the adhesive on the first and second adhesive pads is activated when the first and second adhesive pads contact each other.

Example 5 is an extension of Example 1, or any other example disclosed herein, wherein the clutch mechanism comprises a spring clamp attached to a housing of the drug delivery device and disposed around an outer surface of the outer reservoir, wherein the spring clamp, in a tensioned state allows linear translation of the outer reservoir and, in an un-tensioned state engages the outer reservoir to prevent further translation of the outer reservoir.

Example 6 is an extension of Example 1, or any other example disclosed herein, wherein the clutch mechanism further comprises a guy line attached to a housing of the drug delivery device and to the outer reservoir, the outer reservoir moving along the guy line as the pumping mechanism is being filled, and a spring mechanism which, when un-tensioned, tensions the guy line to prevent further movement of the outer reservoir.

Example 7 is an extension of example 1, or any other example disclosed herein, wherein the clutch mechanism comprises a guy line attached to a housing of the drug delivery device and to the outer reservoir, the outer reservoir pulling the guy line as the pumping mechanism as is being filled, and a clamp mechanism which, when un-tensioned, clamps the guy line to prevent further movement of the outer reservoir.

Example 8 is an extension of Example 1, or any other example disclosed herein, wherein the clutch mechanism comprises one or more bi-stable mechanisms rigidly attached to a housing of the drug delivery device which, in a first stable state, are disengaged from the outer reservoir so as to allow translation of the outer reservoir and, in a second stable state, are engaged with the outer reservoir to prevent further movement of the outer reservoir.

Example 9 is a second embodiment of a pumping mechanism for drug delivery device comprising an outer reservoir, an inner reservoir configured to linearly translate into the outer reservoir, a drive mechanism for linearly translating the inner reservoir into the outer reservoir and a clutch mechanism for coupling the inner reservoir to the drive mechanism.

Example 10 is an extension of Example 9, or any other example disclosed herein, wherein the drive mechanism comprises a tube nut and a leadscrew.

Example 11 is an extension of Example 10, or any other example disclosed herein, wherein the clutch mechanism comprises a torsional spring disposed around an outer surface of the tube nut, wherein the torsional spring, in a tensioned state, allows the leadscrew to linearly translate through the tube nut and, in an un-tensioned state, forces the tube nut into a threaded engagement with leadscrew, wherein the leadscrew is coupled to the inner reservoir.

Example 12 is an extension of Example 10, or any other example disclosed herein, wherein the clutch mechanism comprises a spring, disposed around the tube nut and a collet disposed around the tube nut, wherein the spring, when un-tensioned, translates the collet such that the collet forces the tube nut into a threaded engagement with the leadscrew, wherein the leadscrew is coupled to the inner reservoir.

Example 13 is an extension of claim 10, or any other example disclosed herein, wherein at least a portion of the leadscrew has an oval cross-sectional shape, wherein an inner diameter of the tube nut has an oval cross-sectional shape, wherein, during the filling process, a major axis of the oval portion of the leadscrew and a major axis of the inner diameter of the tube nut are aligned and wherein, after the filling process, the tube nut rotated such that the major axis of the oval portion of the leadscrew is aligned with a minor axis of the inner diameter of the tube nut so as to cause a frictional engagement therebetween, the leadscrew being in the threaded engagement with the inner reservoir.

Example 14 is an extension of Example 10, or any other example disclosed herein, wherein the leadscrew has one or more notched portions along a longitudinal length of the leadscrew, the notched portions being un-threaded, wherein an inner diameter of the tube nut has un-threaded portions corresponding to the unthreaded notched portions of the leadscrew, wherein, after the filling process, rotation of the tube nut creates a threaded engagement with the leadscrew and wherein the leadscrew is coupled to the inner reservoir.

Example 15 is an extension of Example 10, or any other example defined herein, wherein the clutch mechanism comprises a clamp, coupled to the inner reservoir and a mechanism for causing the clamp to engage the leadscrew.

Example 16 is an extension of Example 10, or any other example disclosed herein, wherein the clutch mechanism comprises a spring disposed around the leadscrew and a wedge disposed around the leadscrew, wherein the spring, when un-tensioned, forces the wedge into a portion of the inner reservoir configured to accept a wedge, causing the wedge to engage the leadscrew.

Example 17 is an extension of Example 10, or any other example disclosed herein, wherein the clutch mechanism comprises a spring disposed around an outer surface of the inner reservoir and a clamp, wherein the spring, when un-tensioned, causes the clamp to engage the leadscrew.

Example 18 is an extension of Example 17, or any other example disclosed herein, wherein the clamp is configured with rubber inserts provide a frictional engagement with leadscrew.

Example 19 is an extension of Example 17, or any other example disclosed herein, wherein the leadscrew is configured with the scalloped portion on one end thereof to provide a frictional engagement with the clamp.

Example 20 is extension of Example 10, or any other example disclosed herein, wherein the clutch mechanism comprises a torsional spring coupled at one end to the inner reservoir and disposed around the leadscrew, wherein the torsional spring, in a tensioned state, allows translation of the leadscrew and, in an un-tensioned state, engages the leadscrew, preventing further translation of the leadscrew.

Example 21 is an extension of Example 10, or any other example disclosed herein, wherein the clutch mechanism comprises a clamp coupled to the inner reservoir and disposed around an outer circumference of the tube nut and a spring connected between two ends of the clamp, wherein the spring, in a tensioned state, allows the tube nut to translate through the clamp and, in an un-tensioned state, forces the clamp into engagement with the tube nut, preventing further translation of the tube nut to the clamp.

Example 22 is an extension of Example 10, or any other example disclosed herein, wherein the clutch mechanism comprises a rod, coupled to the inner reservoir and a clamp, coupled to the tube nut, wherein the clamp, in a tensioned state, allows linear translation of the rod and, in an un-tensioned state, prevents linear translation of the rod.

Example 23 is an extension of Example 10, or any other example disclosed herein, wherein the clamp comprises a spring and a collet, wherein the spring, when un-tensioned, forces the collet through a hole defined in the tube nut to engage the rod, thus preventing linear translation of the rod.

Example 24 is an extension of Example 10, or any other example disclosed herein, wherein the clutch mechanism comprises a rod coupled to the inner reservoir and a two-bodied torsional spring, one body of the torsional spring disposed around the rod and another body of the torsional spring disposed around the tube nut, wherein the two-bodied torsional spring, in a tensioned state, allows linear translation of the rod and, in an un-tensioned state, prevents linear translation of the rod.

Software related implementations of the techniques described herein may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

To those skilled in the art to which the invention relates, many modifications and adaptations of the invention may be realized. Implementations provided herein, including sizes, shapes, ratings, compositions and specifications of various components or arrangements of components, and descriptions of specific manufacturing processes, should be considered exemplary only and are not meant to limit the invention in any way. As one of skill in the art would realize, many variations on implementations discussed herein which fall within the scope of the invention are possible. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A pumping mechanism for a drug delivery device comprising:
an outer reservoir;
an inner reservoir configured to linearly translate into the outer reservoir;
a drive mechanism for linearly translating the inner reservoir into the outer reservoir; and
a clutch mechanism for coupling the outer reservoir or the inner reservoir to a housing of the drug delivery device.

2. The pumping mechanism of claim 1 wherein the clutch mechanism comprises:
a brake pad; and
a lever for forcing the brake pad into engagement with an outer surface of the outer reservoir.

3. The pumping mechanism of claim 1 wherein the clutch mechanism comprises:
a first piece of hook and loop fasteners attached to housing of the drug delivery device;
a second piece of mating hook and loop fasteners attached to an outer surface of the outer reservoir; and
a sheath disposed between the first and second pieces of hook and loop fasteners to prevent engagement of the first and second pieces of hook and loop fasteners;
wherein the sheath is removed after the filling of the pumping mechanism to allow engagement of the first and second pieces of mating hook and loop fasteners to prevent further movement of the outer reservoir.

4. The pumping mechanism of claim 1 wherein the clutch mechanism comprises:
a first adhesive pad attached to a housing of the drug delivery device;
a second adhesive pad attached to an outer surface of the outer reservoir; and
a liner disposed between the first adhesive pad and the second adhesive pad;
wherein the liner is removed after the filling of the pumping mechanism and further wherein an adhesive on the first and second adhesive pads is activated when the first and second adhesive pads contact each other.

5. The pumping mechanism of claim 1 wherein the clutch mechanism comprises:
a spring clamp attached to a housing of the drug delivery device and disposed around an outer surface of the outer reservoir;
wherein the spring clamp, in a tensioned state, allows linear translation of the outer reservoir and, in an un-tensioned state, engages the outer reservoir to prevent further translation of the outer reservoir.

6. The pumping mechanism of claim 1 wherein the clutch mechanism comprises:
a guy line attached to a housing of the drug delivery device and to the outer reservoir, the outer reservoir moving along the guy line as the pumping mechanism is being filled; and
a spring mechanism which, when un-tensioned, tensions the guy line to prevent further movement of the outer reservoir.

7. The pumping mechanism of claim 1 wherein the clutch mechanism comprises:
a guy line attached to a housing of the drug delivery device and to the outer reservoir, the outer reservoir pulling the guy line as the pumping mechanism is being filled; and
a clamp mechanism which, when un-tensioned, clamps the guy line to prevent further movement of the outer reservoir.

8. The pumping mechanism of claim 1 wherein the clutch mechanism comprises:
one or more bi-stable mechanisms rigidly attached to housing of the drug delivery device which, in a first stable state, are disengaged from the outer reservoir so as to allow translation of the outer reservoir and, in a second stable state, are engaged with the outer reservoir to prevent further movement of the outer reservoir.

9. A pumping mechanism for a drug delivery device comprising:
an outer reservoir;
an inner reservoir, configured to linearly translate into the outer reservoir;
a drive mechanism for linearly translating the inner reservoir into the outer reservoir; and
a clutch mechanism for coupling the inner reservoir to the drive mechanism.

10. The pumping mechanism of claim 9 wherein the drive mechanism comprises:
a tube nut; and
a leadscrew.

11. The pumping mechanism of claim 10 wherein the clutch mechanism comprises:
a torsional spring disposed around an outer surface of the tube nut;
wherein the torsional spring, in a tensioned state, allows the leadscrew to linearly translate through the tube nut and, in an un-tension state, forces the tube nut into a threaded engagement with the leadscrew; and
wherein the leadscrew is coupled to the inner reservoir.

12. The pumping mechanism of claim 10 wherein the clutch mechanism comprises:
a spring, disposed around the tube nut; and
a collet disposed around the tube nut;
wherein the spring, when un-tensioned, translates the collet, the translation of the collet forcing the tube nut into a threaded engagement with the leadscrew;
wherein the leadscrew is coupled to the inner reservoir.

13. The pumping mechanism of claim 10:
wherein at least a portion of the leadscrew has an oval cross-sectional shape;
wherein an inner diameter of the tube nut has an oval cross-sectional shape;
wherein, during the filling process, a major axis of the oval portion of the leadscrew and a major axis of the inner diameter of the tube nut are aligned; and
wherein, after the filling process, the tube nut rotates such that the major axis of the oval portion of the leadscrew is aligned with a minor axis of the inner diameter of the tube nut so as to cause a frictional engagement therebetween, the leadscrew in threaded engagement with the inner reservoir.

14. The pumping mechanism of claim 10:
wherein the leadscrew has one or more unthreaded notched portions along a longitudinal length thereof;
wherein an inner diameter of the tube nut has one or more unthreaded portions corresponding to the unthreaded notched portions of the leadscrew;
wherein, after the filling process, rotation of the tube nut creates a threaded engagement with the leadscrew; and
wherein the leadscrew is coupled to the inner reservoir.

15. The pumping mechanism of claim 10, the clutch mechanism comprising:
a clamp, coupled to the inner reservoir; and
a mechanism for causing the clamp to engage the leadscrew.

16. The pumping mechanism of claim 15, wherein the clamp is configured with rubber inserts to provide a frictional engagement with the leadscrew.

17. The pumping mechanism of claim 15, wherein the leadscrew is configured with a scalloped portion on one end thereof to provide a frictional engagement with the clamp.

18. The pumping mechanism of claim 10, the clutch mechanism comprising:
a spring disposed around the leadscrew; and
a wedge disposed around the leadscrew;
wherein the spring, when un-tensioned, forces the wedge into a portion of the inner reservoir configured to accept the wedge, causing the wedge to engage the leadscrew.

19. The pumping mechanism of claim 10, the clutch mechanism comprising:
a spring disposed around an outer surface of the inner reservoir; and
a clamp;
wherein the spring, when un-tensioned, causes the clamp to engage the leadscrew.

20. The pumping mechanism of claim 10, the clutch mechanism comprising:
a torsional spring coupled at one end to the inner reservoir and disposed around the leadscrew;
wherein the torsional spring, in a tensioned state, allows translation of the leadscrew and, in an un-tensioned state, engages the leadscrew, preventing further translation of the leadscrew.
